(19) United States
(12) Patent Application Publication
(10) Pub. No.: US 2021/0038563 A1
(43) Pub. Date: Feb. 11, 2021

(54) NEW SPIRONOLACTONE FORMULATIONS AND THEIR USE

(71) Applicants: Apidel SA, Geneva (CH); Inserm, Paris (FR); SORBONNE UNIVERSITE, Paris (FR); UNIVERSITE de Paris, Paris (FR); Assistance Publique, Paris (FR)

(72) Inventors: Robert GURNY, Geneva (CH); Francine BEHAR-COHEN, Paris (FR); Jean Louis BOURGES, Paris (FR)

(21) Appl. No.: 16/964,263

(22) PCT Filed: Jan. 24, 2019
(86) PCT No.: PCT/EP2019/051764

§ 371 Date: Jul. 23, 2020

(30) Foreign Application Priority Data
Jan. 26, 2018 (EP) 18000064.8

(51) Int. Cl. A61K 31/365; A61K 47/34; A61K 9/107; A61P 27/02

(52) U.S. Cl. CPC A61K 31/365; A61P 27/02; A61K 9/1075; A61K 47/34

(57) ABSTRACT

The invention relates to a pharmaceutical formulation comprising a spironolactone and at least one polymer or a polymer mixture as well as its use in particular indications.

The invention relates to a pharmaceutical formulation comprising a spironolactone and at least one polymer or a polymer mixture as well as its use in particular indications and method of making.

### BACKGROUND

Spironolactone is known for various medical applications and indications. Unfortunately, the systemic medical use of spironolactones implies a number of unwanted side effects.

Polymers or polymer mixtures like an alkyl substituted polylactide or/and a polymer prepared by melt polycondensation of one or more substituted or unsubstituted C₆-C₈ 2-hydroxyalkyl acid(s), as well as block co-polymers of these compounds with methoxypoly(ethylene glycol) (mPEG), are known from WO2007/012979 A1 and WO2012/014011 A1.

Also spironolactone formulations and their medical use are known, however, also the known medical formulations comprising spironolactone imply various disadvantages.

Wound healing also implies challenges and it is by far a medical issue solved by modern medicine. In fact impaired wound healing is a significant clinical problem encountered as a complication of certain chronic conditions such as diabetes, sickle cell disease, Cushing syndrome and in patients receiving prolonged glucocorticoid therapy [1, 2]. Impaired corneal wound healing is a major concern in ophthalmology since it can cause corneal opacity and scarring leading to major visual disturbance, chronic infection and ulceration and may ultimately lead to loss of sight (corneal blindness) [3, 4].

Wound healing is a complex and highly organized process that encompasses successive and overlapping stages including inflammation, granular tissue formation and re-epithelialization, new matrix formation and collagen accumulation. The whole process is tightly controlled by a precise and complex interplay of various factors involving cells, growth factors, cytokines and components of the extracellular matrix [1, 2, 5-8]. Whilst wound healing follows a uniform pattern all over the body, local specificities exist resulting from tissue-specific differences, for example, the lack of blood vessels in the cornea compared to the skin [8].

The critical feature of wound healing is the restoration of the epithelial barrier. Re-epithelialization in the cornea is a key step in preventing abnormal healing and subsequent impaired vision [6]. During re-epithelialization, corneal epithelial cells proliferate at the wound edge, migrate to cover the lesioned area and differentiate to form the new tissue. Absence of keratinocyte migration is related to the clinical phenotype of chronic non-healing wounds, e.g. diabetic ulcers. When total re-epithelialization is achieved, the barrier is restored and the eye is again protected from external infections [2, 3, 5, 6, 8].

Synthetic glucocorticoids (GC) are among the most widely prescribed drugs in the world. They are given systemically or topically to treat a wide number of inflammatory and autoimmune diseases, allergies and ocular disorders. In ophthalmology, GC are currently used to prevent and to treat post-operative ocular inflammation, corneal graft rejection, corneal neovascularization, ocular infections and they are also indicated for the treatment of many ocular surface disorders including dry eye [2, 7, 9, 10].

Whilst the pleiotropic anti-inflammatory effects of GC reduce cytotoxic and pro-angiogenic cytokines and metalloproteinase expression [11], they are also associated with delayed epithelial healing [2, 8, 12]. Several in vivo studies have reported that the use of GC such as dexamethasone resulted in delayed corneal wound healing in rabbits [4, 7, 13, 14]. More significantly, GC treatment also leads to reduced and delayed wound re-epithelialization in humans [15]. Results from a clinical trial including 42 patients who received topical prednisolone phosphate showed that they re-epithelialized more slowly than the placebo group [4].

GC bind to the glucocorticoid receptor (GR), but they can also bind with high affinity to the closely related mineralocorticoid receptor (MR)—both receptors are expressed in the corneal epithelium. Recent studies reported that in the skin, delayed wound healing might be due to occupancy of the MR by GC. In mineralocorticoid-sensitive tissues such as in the kidney, GC are inactivated by 11b-hydroxysteroid dehydrogenase type II (HSD2), thereby preventing their binding to MR which is therefore selectively activated by aldosterone, the endogenous mineralocorticoid (MC) which binds to the MR and is responsible for sodium homeostasis [2, 9, 12, 16-18]. However, tissues where HSD2 activity is low such as skin, eye, heart, and neurons are susceptible to off-target GC binding to the MR. Given that the MR might be over-activated by GC in tissues where HSD2 activity is low, the use of MR antagonists (MRA) was proposed as a potential therapeutic strategy to overcome the negative impact of GC treatment on wound healing. This hypothesis was verified in several studies: (i) in cultured human skin explants where clobetasol-induced epidermal atrophy was significantly limited by the MR antagonists, potassium canrenoate and eplerenone [2, 9], (ii) in mice where potassium canrenoate significantly improved clobetasol-induced delayed wound healing [2] and (iii) in healthy volunteers, where local co-administration of the MR antagonist, spironolactone, with clobetasol significantly improved the clobetasol-induced impairment of skin wound closure [9].

Finally, spironolactones are so far administered systemically which involves undesirable side-effects. Moreover; the ocular bioavailability of spironolactone is very low since spironolactone is a known target of efflux proteins. Thus, spironolactone can only by used systemically if ocular barriers are compromised or in conditions where the primary site of disease if the vascular endothelium. In other ocular diseases, in which the MR must be targeted in ocular cells, the systemic administration of spironolactone is not efficient. [33].

Moreover, the unwanted side-effects are significant, e.g. effects on fertility in women and feminization (such as development of breast tissue) in men is documented.

Accordingly, it was an object of the present application to provide for a pharmaceutical formulation which does not exhibit the unwanted side-effects of spironolactone and/or known formulations, or at least to reduce the known side-effects of spironolactone and/or known spironolactone formulations.

The formulation aims at targeting optimally directly ocular tissues that cannot be efficiently targeted by the systemic use of spironolactone due to the ocular barriers.

It was another object of the present application to provide for a pharmaceutical formulation which can be delivered directly to target tissues and thereby avoids unwanted side effects of spironolactone.

### BRIEF SUMMARY OF THE DISCLOSURE

In one aspect the disclosure relates to a pharmaceutical formulation comprising a spironolactone and at least one polymer or a polymer mixture wherein the polymer or a polymer mixture is selected from one or several as disclosed in WO2007/012979 A1 and WO2012/014011 A1.

In another aspect the disclosure relates to a pharmaceutical formulation suited to the local or regional administration of the formulation.

In yet another aspect the disclosure relates to a method of preventing, repressing or treating a disease or disorder selected from the group comprising an ophthalmic disease or disorder, or a skin disease or disorder, or related diseases or disorders.

In yet another aspect the disclosure relates to a pharmaceutical formulation for use in the preventing, repressing or treating a disease or disorder selected from the group comprising an ophthalmic disease or disorder.

In yet another aspect the disclosure relates to a method for preparing a pharmaceutical composition.

In yet another aspect the disclosure relates to a formulation for use wherein it is used in patients with prior or concomitant treatment of corticosteroids or corticosteroid medication.

### BRIEF DESCRIPTION OF THE FIGURES

FIG. 1: Transmission electron microscopy (TEM) image of 0.1% spironolactone loaded micelles showing their spherical shape and homogeneity.

FIG. 2: Mean percentage re-epithelialization of the corneal wounds per treatment group after 4-days' treatment. Bars represent means, errors bars represent standard deviation. p-values were calculated using Kruskal-Wallis one-way analysis of variance on ranks followed by Student-Newman-Keuls post-hoc analysis test; ns (p>0.05), non-significant difference, * (p<0.05), significant difference.

FIG. 3 A—D: Mean concentrations of the drugs found in the right treated and left control corneas after 5-days multiple instillation of A, 0.1% spironolactone micelles followed by dexamethasone (n=10); B, 0.01% spironolactone micelles followed by dexamethasone (n=9); C, 0.1% potassium canrenoate solution followed by dexamethasone (n=10); D, dexamethasone (n=10). p-values are obtained with Student t-test; ns, p>0.99.

FIG. 4: Typical SIR traces obtained from rabbit #6 treated with 0.1% SPL-Micelles and Maxidex® (Group 1). A, right treated cornea. B, left control (untreated) cornea. Chromatograms are obtained from the UHPLC-MS analysis of the treated and control corneas of the rabbits involved in the study. Group 1: 0.1% spironolactone micelles+Maxidex®.

FIG. 5: Typical SIR traces obtained from rabbit #20 treated with 0.01% SPL-Micelles and Maxidex® (Group 2). A, right treated cornea. B, left control (untreated) cornea. Group 2: 0.01% spironolactone micelles+Maxidex®

FIG. 6: Typical SIR traces obtained from rabbit #25 treated with 0.1% potassium canrenoate solution and Maxidex® (Group 3). A, right treated cornea. B, left control (untreated) cornea.

FIG. 7: Typical SIR traces obtained from rabbit #38 treated with PBS (Group 4). A, right treated cornea. B, left control (untreated) cornea.

FIG. 8: Typical SIR traces obtained from rabbit #42 treated with Maxidex® (Group 5). A, right treated cornea. B, left control (untreated) cornea.

### DETAILED DESCRIPTION OF THE DISCLOSURE

In the following the different aspects of the disclosure will be described in more detail which is not to be understood as limiting of the invention but referring to variations and possibly preferred embodiments which may include additional variations which will be apparent to the skilled person.

In the context of the present disclosure “spironolactone” may be used in any known form, and it is also denoted SC-9420; NSC-150339; 7α-Acetylthiospirolactone; 7α-Acetylthio-17α-hydroxy-3-oxopregn-4-ene-21-carboxylic acid γ-lactone) as well as tautomers, geometrical isomers, optically active forms, enantiomeric mixtures thereof, pharmaceutically acceptable salts and pharmaceutically active derivative thereof.

A “polymer or a polymer mixture” according to the disclosure is used as defined below and as described in WO2007/012979 A1 and/or in WO2012/014011 A1 which is incorporated by reference herein. In particular, the “polymer or polymer mixture” according to the disclosure is a co-polymer of mPEG and poly(caprylic acid).

Poly(caprylic acid) according to the disclosure is a homopolymer of caprylic acid prepared by any polymerization method known in the art. Caprylic acid is the common name for the eight-carbon saturated fatty acid known by the systematic name octanoic acid. Caprylic acid has a GRAS (“generally recognized as safe”) status and has been designated E570 in the European food safety database.

Poly(caprylic acid) is also known variously as poly-hydroxy octanoic acid (“polyHOA”) and as hexyl-substituted poly lactic acid (“hexPLA”)

An “indication or formulation for use” is defined below and may refer to any ophthalmic uses or used for protecting or treating epithelial tissue, in particular the cornea and corneal tissue.

A “pharmaceutical formulation” in the sense of the disclosure is as follows: Pharmaceutical compositions of the present invention comprise an effective amount of spironolactone, together with one or more alkyl substituted polylactide or additional agent(s) dissolved in or dispersed in, a pharmaceutically acceptable carrier. Further it is recognized that one or more alkyl substituted polylactide may be used in combination with an additional agent in or as a pharmaceutically acceptable carrier.

The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one alkyl substituted polylactide or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The formulation of the present disclosure can be preferably administered locally, or by any method or any combination of the forgoing as would be known to one of ordinary skill in the art (see, for example, Remington's Pharmaceutical Sciences, 18th Ed. Mack Printing Company, 1990, incorporated herein by reference).

Upon formulation, solutions will be administered in a manner compatible with the dosage formulation and in such amount as is therapeutically effective. The formulations are easily administered in a variety of dosage forms.

Further in accordance with the present invention, the composition of the present invention suitable for administration is provided in a pharmaceutically acceptable carrier with or without an inert diluent.

In accordance with the present invention, the composition is combined with the carrier in any convenient and practical manner, i.e., by solution, suspension, emulsification, admixture, encapsulation, absorption and the like. Such procedures are routine for those skilled in the art.

Stabilizing agents can be also added in the mixing process in order to protect the composition from loss of therapeutic activity, Examples of stabilizers for use in an the composition include buffers, pH regulators, antioxidants, amino acids such as glycine and lysine, carbohydrates such as dextrose, mannose, galactose, fructose, lactose, sucrose, maltose, sorbitol, mannitol, etc.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according tot he response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.001% of an active compound. In other embodiments, the active compound may comprise between about 0.1% to about 25.0% of the weight of the unit, or between about 0.5% to about 10%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Polylactides are known in the art. For example, U.S. Pat. Nos. 6,469,133, 6,126,919 describe various polylactides and are incorporated by reference herein in their entirety without disclaimer. Polylactides are biodegradable which enhances their utility. For example, polylactides may be degraded in the body of a subject (e.g., a human patient) into the constituent hydroxycarboxylic acid derivatives (i.e. lactic acids) that form over a period of weeks or years. Polylactides can have molecular weights from about 2000 Da to about 250,000 Da. For these reasons, polylactides may be attractive materials for generating items such as degradable sutures, pre-formed implants, and compounds for drug delivery (e.g., sustained release matrices).

In one aspect the disclosure relates to a pharmaceutical formulation comprising a spironolactone (also denoted: SC-9420; NSC-150339; 7α-Acetylthiospirolactone; 7α-Acetylthio-17α-hydroxy-3-oxopregn-4-ene-21-carboxylic acid γ-lactone) as well as tautomers, geometrical isomers, optically active forms, enantiomeric mixtures thereof, pharmaceutically acceptable salts and pharmaceutically active derivative thereof and at least one polymer or a polymer mixture of one or more of an alkyl substituted polylactide or/and a polymer prepared by melt polycondensation of one or more substituted or unsubstituted C₆-C₈ 2-hydroxyalkyl acid(s), such polymer(s) being a co-polymer of a polymer of 2-hydroxyl acid(s) with mPEG.

It has been found that the disadvantage of known formulations and the use of spironolactone in known applications could be overcome with the pharmaceutical formulation according to the disclosure.

The new formulations according to the disclosure provide for and avoid unwanted side-effects of spironolactone by the provision of a formulation which may be applied topically on the ocular surface. Moreover, corticosteroid treatment side-effects can now be avoided.

The pharmaceutical formulation according to the disclosure can be used in any suitable manner and as is the usual practice in the medical field and in the context of pharmaceutical formulations. It is possible to prepare the pharmaceutical formulation according to the disclosure in a manner wherein the formulation is sterile filtered.

The pharmaceutical formulation according to the disclosure exhibits advantageous characteristics in various aspects. In particular the pharmaceutical formulation according to the disclosure provides for improved tissue penetration characteristics of spironolactone.

The inventive pharmaceutical formulation and in particular the usefulness thereof in topical applications is advantageous as it is now possible to treat certain indications with spironolactone without the risk of the known unwanted side-effects in an efficient way and to treat corneal diseases that cannot be efficiently targeted by the systemic use of spironolactone.

The pharmaceutical formulation according to the disclosure provides an advantageous combination with the at least one polymer or/and the polymer mixture. Particularly advantageous is that by the spontaneous encapsulation of the spironolactone within very small micellar structures formed from the co-polymers of the 2-hydroxyalkyl acid(s) with mPEG, a clear aqueous formulation of the water insoluble spironolactone drug may be prepared. Additionally, the hydrophilic shells of such micellar structures may advantageously interact intimately with naturally-hydrated tissue surfaces. Even more advantageously, the greatly enhanced surface area of drug-loaded micellar structures facilitates rapid and efficient transfer of drug into the tissue onto which the formulation is administered.

The advantageous characteristics of the pharmaceutical formulation according to the disclosure is partly or entirely due to the polymer which is selected from one or more of
a. one or more of a co-polymer consisting of mPEG and an alkyl substituted polylactide, and wherein the alkyl substituted polylactide is viscous and has the structure:

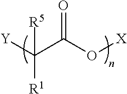

wherein R¹ is substituted or unsubstituted C₂-C₃₀ alkyl, wherein n is at least 2: and wherein R³ is hydrogen or substituted or unsubstituted alkyl. In specific aspects, the polymer can be a polymer of any one or more of the C₄-C₃₂ 2-hydroxylalkyl acids: wherein X is hydrogen or —C(O)—CH—CH2; and Y is selected from the group consisting of —OH, an alkoxy, benzyloxy and —O—(CH2-CH2-O)p-CH3; and wherein p is 1 to 700 and as disclosed in WO2007/012979 A1
and/or
b. one or more polymers prepared by melt polycondensation of one or more substituted or unsubstituted C₆-C₈ 2-hydroxyalkyl acid(s) and as disclosed in WO2012/014011 A1.

Particularly advantageous is a pharmaceutical formulation according to the disclosure wherein the active compound is a spironolactone and the polymer is a co-polymer consisting of mPEG and poly(caprylic acid).

One advantage of said formulation is the fact that it can be administered for ocular applications without the risk of impairing the visibility of a patient in view of the fact that the formulation is clear and does not physically obstruct the vision of the patient.

The pharmaceutical formulation according to the current disclosure can advantageously be applied for use in the preventing, repressing or treating a disease or disorder selected from the group comprising an ophthalmic disease or disorder, recurrent corneal erosions, wound healing delay particularly but not only due to association with the use of glucocorticoids, post surgical treatment of corneal graft to favor re-epithelialization, glucocorticoid topical administration on desepithelialized cornea such as cornea traumatism, post corneal surgery, post cross-linking, post refractive surgery (laser assisted or surgical procedure), corneal dystrophies, ocular rosacea, corneal abscess or any bacterial infection in association with antibiotics, corneal fibrosis and scaring due to anti-fibrotic effects of spironolactone, corneal opacification, peripheral ulcerative keratitis, corneal neovascularization (e.g. due to anti-angiogenic effects of spironolactone), meibomian gland dysfunction and associated diseases such as dry eye syndromes and blepharitis.

The inventive pharmaceutical formulation can be used for applications and indications, respectively, as described above. It will also be appreciated by the skilled person that it is also feasible to use said formulation for treating any epithelial tissue and/or skin wherein the similar receptor compositions occur as in the eye, or/and wherein similar or comparable target receptors occur.

The pharmaceutical formulation according to the applications and use as describe above will exhibit a number of advantages. Said pharmaceutical formulation in particular is characterized by a reduced incidence of side effects, while maintaining at least an equivalent efficacy to known treatments

Another aspect of the disclosure is a method of preventing, repressing or treating a disease or disorder selected from the group comprising an ophthalmic disease or disorder, or a skin disease or disorder, recurrent corneal erosions, wound healing delay associated with the use of glucocorticoids, post surgical treatment of corneal graft to favor reepithelialization, glucocorticoid topical administration on desepithelialized cornea such as cornea traumatism, post corneal surgery, post cross-linking, post refractive surgery (laser assisted or surgical procedure), corneal dystrophies, ocular rosacea, corneal abscess and bacterial infections in association with antibiotics, corneal fibrosis and scaring due to anti-fibrotic effects of spironolactone, corneal opacification in a subject said method comprising administering to a subject in need thereof a pharmaceutical formulation as disclosed herein.

Another aspect of the disclosure is a method for treating or preventing an ophthalmic disease or disorder associated with excessive stimulation of the mineralocorticoid receptor by administering a pharmaceutical formulation as disclosed herein.

Another aspect of the disclosure is a method for treating an ophthalmic disease or disorder wherein the stimulation is engendered by gluco corticosteroid therapy by administering a pharmaceutical formulation as disclosed herein.

Another aspect of the disclosure is a method for treating an ophthalmic disease or disorder wherein the disease or disorder is selected from a disease or disorder selected from the group comprising an ophthalmic disease or disorder, recurrent corneal erosions, wound healing delay particularly but not only due to association with the use of glucocorticoids, post surgical treatment of corneal graft to favor re-epithelialization, glucocorticoid topical administration on desepithelialized cornea such as cornea traumatism, post corneal surgery, post cross-linking, post refractive surgery (laser assisted or surgical procedure), corneal dystrophies, ocular rosacea, corneal abscess and any corneal bacterial infection association with antibiotics, corneal fibrosis and scaring due to anti-fibrotic effects of spironolactone, corneal opacification, peripheral ulcerative keratitis, corneal neovascularization (due to anti-angiogenic effects of spironolactone), meibomian gland dysfunction and associated diseases such as dry eye syndroms and blepharitis by administering a pharmaceutical formulation as disclosed herein.

Another aspect of the disclosure is a method for preparing a pharmaceutical composition as disclosed herein by mixing the two components at room temperature.

Another aspect of the disclosure is a formulation for use as disclosed herein, or the method as disclosed herein for topical use or administration, or for a loco-regional use or administration.

Another aspect of the disclosure is a formulation for use as disclosed herein, or a method as disclosed herein wherein it is used in patients with prior or concomitant treatment of corticosteroids or corticosteroid medication.

### EXAMPLES

The Examples will illustrate various aspects of the disclosure and the current inventive aspects without meant to being understood as restrictive in any manner.

The Examples will inter alia illustrate the disclosure and the inventive formulations and their use in the context of glucocorticoid use, corneal wound healing, reduction of spironolactone side effects, polymeric nanocarriers as advantageous formulation component, pre-clinical in vivo tolerability and efficacy aspects of the inventive formulations as described above.

The objective of the following Examples was inter alia to investigate whether mineralocorticoid receptor antagonism using a topical micellar formulation of spironolactone could prevent glucocorticoid-induced delayed corneal wound healing in New Zealand white rabbits. Spironolactone micelles (0.1% w/v) with a mean number weighted diameter of 20 nm were prepared using mPEG-hexPLA (mPEG-poly(caprylic acid) polymer and shown to have a midterm stability of at least 6 months at 5° C. Preclinical studies in New Zealand white rabbits demonstrated that the 0.1% spironolactone micellar formulation was well-tolerated since no reaction was observed in the cornea following multiple daily instillation over 5 days. The preclinical studies also confirmed that dexamethasone significantly delayed epithelial wound healing as compared to untreated control (percentage re-epithelialization after Day 4: 84.6±13.9% versus 99.5±1.0%, p<0.05). However, the addition of the 0.1% spironolactone micellar formulation significantly improved the extent of re-epithelialization, countering the dexamethasone induced delayed wound healing with a percentage re-epithelialization that was statistically equivalent to the untreated control (96.9±7.3% versus 99.5±1.0%, p>0.05). The biodistribution study provided insight into the ocular metabolism of spironolactone and hence the relative contributions of the parent molecule and its two principal metabolites, 7α-thiomethylspironolactone and canrenone, to the observed pharmacological effects. Comparison of the efficacies of spironolactone and potassium canrenoate (a water-soluble precursor of canrenone) in overcoming the dexamethasone-induced delayed wound healing confirmed that the former had greater efficacy. The results pointed to the greater potency of 7α-thiomethylspironolactone over canrenone as a mineralocorticoid receptor antagonist which explained its superior ability in countering the glucocorticoid-induced over-activation that was responsible for the delayed wound healing. In conclusion, the preliminary results supported the above-mentioned hypothesis suggesting that co-administration of mineralocorticoid receptor antagonists to patients under glucocorticoid therapy might prevent the deleterious effects of glucocorticoids on complex corneal wound healing processes.

One objective of the following examples was to investigate whether, as in the case of the skin, GC-induced delayed corneal wound healing could be reversed by MR antagonists. To test this hypothesis, a novel micellar formulation of the potent MR antagonist spironolactone (0.1%, w/v) was developed and characterized for topical ocular administration and then evaluated to determine whether it was possible to counter the impaired corneal wound healing induced by dexamethasone in New Zealand white rabbits. It was decided to compare the results to those observed after topical application of a lower concentration micellar formulation of spironolactone (0.01%, w/v) and a formulation containing the water-soluble prodrug, potassium canrenoate (0.1%, w/w), which is a precursor of canrenone, a pharmacologically active metabolite of spironolactone.

### 1. MATERIAL AND METHODS

2.1. Materials

Methoxy-poly(ethylene glycol)-hexyl-substituted-poly(lactic acid), (mPEG-hexPLA, 5.5 kDa) was supplied by Apidel SA (Geneva, Switzerland). Spironolactone (SPL) was purchased from Zhejiang Langhua pharmaceutical Co., Ltd. (Zhejiang, China). 7α-thiomethylspironolactone (TMSPL) was purchased from TLC Pharmaceutical Standards Ltd. (Ontario, Canada). Canrenone (CAN), potassium canrenoate (CANK) and 17α-methyltestosterone (MeT), used as an internal standard (IS), were purchased from Sigma-Aldrich (Buchs, Switzerland). Dexamethasone (DXM) was purchased from Tianjin TianMao Technology Development Corp. Ltd (Tianjin, China). Maxidex® (dexamethasone 0.1% suspension, Alcon) was purchased from a local pharmacy. Sodium chloride was obtained from Hänseler AG (Herisau, Switzerland). Ultrapure water (H₂O) was prepared using a Merck Millipore Milli-Q water purification system (Darmstadt, Germany) (resistivity >18 MΩ cm). Methanol (MeOH, HPLC grade) was obtained from Fisher Scientific (Waltham, Mass., USA), acetonitrile (ACN, HPLC grade) and formic acid (ULC/MS grade) from Biosolve (Dieuze, France). Acetone Chromasolv® (HPLC grade) was purchased from Sigma Aldrich (Buchs, Switzerland) and trifluoroacetic acid was obtained from VWR (Dietikon, Switzerland). All other chemicals were at least of analytical grade.

Millex® filters (Durapore PVDF, pore size 0.22 μm, diameter 13 mm) were purchased from Sigma-Aldrich (Buchs, Switzerland). 10 mL sterile eye drop vials were purchased from Müller+Krempel AG (Bülach, Switzerland).

2.2. Methods

2.2.1. Analytical Methods

2.2.1.1. HPLC Methods

HPLC analytical methods were developed to support the formulation development and stability study of both spironolactone micelles and the potassium canrenoate solution. Quantification of spironolactone by HPLC-UV: Spironolactone quantification was performed on an Agilent 1100 HPLC using a reversed phase column (YMC basic, 250×3.0 mm, 5 μm) heated to 40° C. The method employed a gradient of acetonitrile and water containing 0.1% trifluoroacetic acid: the acetonitrile percentage was increased from 40% to 80% within 5 min, kept constant for 3 min and then decreased to 40% within half a minute. The mobile phase flow rate was 1.0 mL/min and the UV detector was set to 238 nm.

Quantification of potassium canrenoate by HPLC-UV: Potassium canrenoate quantification was performed on an Agilent 1100 HPLC using a reversed phase column (YMC basic, 250×3.0 mm, 5 μm) heated to 40° C. The mobile phase consisted of acetonitrile containing 0.1% trifluoroacetic acid (A) and water containing 0.1% trifluoroacetic acid (B). The analysis was carried out in isocratic mode with 55% eluent A and 45% eluent B. The mobile phase flow rate was 1.0 mL/min and the UV detector was set to 286 nm.

2.2.1.2. UHPLC-MS Method

A validated UHPLC-MS analytical method (manuscript submitted) was used to quantify the biodistribution of the different analytes in the rabbit corneas obtained from the in vivo study. Briefly, the liquid chromatographic system consisted of a Waters Acquit® ultra performance liquid chromatography (UPLC®) system (Baden-Dättwil, Switzerland) including a binary solvent manager, a sample manager with an injection loop volume of 10 μL and a column manager. The reversed phase chromatographic separation of the six compounds was performed on a Waters XBridge® BEH C18 column (50×2.1 mm I.D., 2.5 μm) fitted with a Waters XBridge® BEH C18 Vanguard pre-column (5×2.1 mm I.D., 2.5 μm). The elution was carried out in isocratic mode with a mobile phase consisting of 0.1% formic acid in H₂O/MeOH (48/52, v/v) with a flow rate of 0.45 mL/min and a run time of 5 min. Column temperature was held at 40° C. and sample manager temperature was kept at room temperature. Injection volume was set at 5 μL. The mass spectrometry (MS) system consisted of a Waters XEVO® TQ-MS detector (Baden-Dättwil, Switzerland) fitted with a Z-spray electrospray ionisation source. MS detection of the six compounds was performed using electrospray ionisation in the positive mode (ESI+) and selected ion recording (SIR) using the pseudo-molecular ion of each compound as the parent ion (hydrogen adduct, [M+H]⁺). The capillary voltage was set at 2.3 kV, and desolvation gas temperature and flow were maintained at 350° C. and 650 L/h, respectively. The specific MS parameters for each analyte were tuned and determined by infusing each compound individually at 1 μg/mL in MeOH:H₂O (1:1) at a flow rate of 5 μL/min. Identification and quantification of each analyte were carried out according to the mass-to-charge ratio (m/z) of the pseudo-molecular ion of each compound (hydrogen adduct, [M+H]⁺). Cone voltage optimal settings were 15 V for DXM, 32 V for CANK and 35 V for SPL, TMSPL, CAN and MeT. The pseudo-molecular parent ion corresponding to DXM, CANK, SPL/CAN, TMSPL and MeT have an m/z of 393.1, 359.1, 341.0, 389.0 and 303.0 respectively. Dwell time was set at 5 ms for all the compounds except for DXM at 328 ms. Data processing was performed using Waters MassLynx software version 4.1 (Baden-Dättwil, Switzerland).

Calibration standards at 10, 20, 50, 100, 200, 500 and 1000 ng/mL were prepared in a corneal matrix obtained from porcine corneas extracted in MeOH:H₂O (1:1). All calibration curves were linear (r²>0.99). The limit of detection (LOD) and the limit of quantification (LOQ) for each analyte are summarized in Table 1.

TABLE 1
LOD and LOQ of each analyte in corneal matrix.

| Analyte | LOD (ng/mL) | LOQ (ng/mL) |
|---|---|---|
| Dexamethasone | 5.4 | 16.3 |
| Potassium canrenoate | 2.4 | 7.2 |
| Spironolactone | 3.8 | 11.4 |
| 7α-thiomethyspironolactone | 1.3 | 3.9 |
| Canrenone | 2.9 | 8.8 |

2.2.2. Development and Optimization of Spironolactone Micellar Formulation

Spironolactone loaded micellar nanocarriers (0.1%, w/v) were prepared using mPEG-hexPLA copolymer at different SPL:copolymer ratios; 1:20, 1:40 and 1:60. Two buffers were also evaluated; citrate buffer (10 mM, pH 5.5) and PBS (10 mM, pH 7.4). Formulations were prepared at a batch size of 10 mL. Briefly, 10 mg spironolactone were dissolved in 2 mL acetone. Then 200, 400 or 600 mg mPEG-hexPLA, corresponding respectively to 1:20, 1:40 and 1:60 SPL:copolymer ratios, were added to the acetone solution containing SPL and dissolved. Subsequently, this solution was added dropwise using a syringe pump (6 mL/h) and under sonication (20% amplitude—S 450 D, Branson, USA) to 10 mL of the aqueous phase, consisting of either citrate buffer (10 mM, pH 5) or PBS (10 mM, pH 7.4). Then, acetone was removed under reduced pressure (58° C., 180 mbar—Buchi Rotavapor R-210; Switzerland). Finally, the osmolarity was adjusted to 270-300 mOsm with NaCl and the formulations were filtered through 0.22 μm PVDF filters into sterilized vials and kept at 5° C. Formulations were characterized in terms of concentration, drug loading, incorporation efficiency and micelles size. Micelles were also visualized using transmission electron microscope (TEM, FEI Tecnai™ G2 Sphera, Oregon, USA). Briefly, the micellar formulation was diluted 1:10 in MilliQ water, then 5 μL were deposited on a grid, left for 30 seconds and the excess was carefully wiped. Subsequently, one drop of 2% uranyl acetate was applied during 30 seconds to enhance the contrast and the excess was carefully removed. TEM magnification was set at 25000×.

2.2.2.1. Determination of Drug Content and Incorporation Efficiency

Spironolactone content was quantified by HPLC-UV. SPL micelles aliquots were diluted with acetonitrile (1:10) prior to HPLC analysis. The drug content and incorporation efficiency were calculated using the following equations:

$$\begin{matrix}Drug Loading ( mg \text{/} g ) = \frac{mass of spironolactone in the formulation ( mg )}{mass of polymer in the formulation ( g )} & Eq . 1 \\ Incorporation Efficiency ( \% ) = \frac{Actual drug loading}{Target drug loading} 100 & Eq . 2\end{matrix}$$

2.2.2.2. Size Determination

The intensity weighted (Z-average) and the number weighted (dₙ) hydrodynamic diameters and the polydispersity index (PDI) of the micelles were measured using a Zetasizer Nano-ZS (Malvern Instruments, UK). SPL micellar solutions were diluted 1:1 in MilliQ water and filled into disposable plastic cuvettes for analysis with back scattering light (173 degrees).

2.2.3. Preparation and Characterization of the Formulations Used in the In Vivo Study

2.2.3.1. Spironolactone Micellar Formulations (0.1% and 0.01%, w/v)

Spironolactone loaded micelles (0.1%, w/v) were prepared at a batch scale of 14 mL. Briefly, 616 mg mPEG-hexPLA and 15.4 mg spironolactone were dissolved in 2 mL of acetone. The organic phase was added dropwise (6 mL/h) to the aqueous phase (10 mM citrate buffer, 0.7% NaCl, pH 5.5) under sonication (20% amplitude—S 450 D, Branson, USA). Subsequently, acetone was removed under reduced pressure (58° C., 180 mbar—Buchi Rotavapor R-210, Switzerland). This formulation was prepared with 10% excess (by weight) to counterbalance the amount of SPL and mPEG-hexPLA lost in the syringe during the formulation process. The 0.01% (w/v) SPL concentration was obtained by 1:10 dilution of the 0.1% SPL micelles in the aqueous phase. Finally, formulations were filtered through 0.22 μm PVDF filters and stored in sterile eye drop vials. Spare aliquots from both formulations were kept to evaluate formulation stability over time.

2.2.3.2. Potassium Canrenoate Solution (0.1% w/w)

Potassium canrenoate solution (0.1% w/w) was prepared by dissolving 50 mg potassium canrenoate in 50 g of aqueous buffer (5 mM phosphate buffer, 0.9% NaCl, pH 8.0). This solution was filtered through 0.22 μm PVDF filters and stored in sterile eye drop vials. Spare aliquots were kept for the stability testing of the formulation over time.

2.2.4. In Vivo Tolerability and Efficacy Study in Rabbits

2.2.4.1. Animals

Fifty male albino New Zealand rabbits weighing approximately 2.3-3.0 kg were included for this study (Iris Pharma, France). Animals were housed individually in standard cages, under identical environmental conditions. The temperature was kept at 15-21° C. and the relative humidity was >45%. Rooms were continuously ventilated (>15 air volumes per hour). Temperature and relative humidity were continuously controlled and recorded. Animals were routinely exposed (in-cage) to a 10-200 1× light in a 12-hour light/dark cycle (from 7:00 a.m. to 7:00 p.m.). Animals had enrichment and free access to food (150 g/day) and were allowed water ad libitum. All animals were healthy and free of clinically observable ocular abnormalities throughout the study. All animals were treated according to the Directive 2010/63/EU—The European convention on the protection of animals used for scientific purposes—and to the Association for Research in Vision and Ophthalmology (ARVO) Statement for the use of animals in ophthalmic and visual research and were approved by the local veterinary authority for animal experimentation (French governmental platform APAFIS—authorization number 20160212659386).

2.2.4.2. Induction of Corneal Wounds

The animals were anesthetized by an intramuscular injection of a ketamine-xylazine mixture. Then, a drop of 0.4% oxybuprocaine was topically applied for local anesthesia. In addition, buprenorphine (20 μg/kg) was administrated by subcutaneous injection 30 min prior to induction to prevent pain. A scalpel handle was used to keep the right eye out of orbit and the corneal epithelium was then completely removed using a scalpel blade. De-epithelialization was monitored by fluorescein staining. Eyes were washed with physiological saline and swabbed with a dry cotton tip applicator to remove cellular debris and re-washed in saline solution.

2.2.4.3. Study Design

Animals were randomized into 5 treatment groups as presented in Table 2. Each group included 10 rabbits and were instilled using an eye-dropper in the right eye 3 times daily on Day 0, 6 times daily from Day 1 to Day 4 and once on Day 5.

Group 1, 2 and 3: Animals were treated with the test items (0.1% spironolactone micelles, 0.01% spironolactone micelles and 0.1% potassium canrenoate solution, respectively)—1 drop (˜35 μL) of the test item then, 5 minutes later, 1 drop of 0.1% dexamethasone (Maxidex®).

Group 4 (positive control group): Animals were treated with the control item (PBS)—2 drops of PBS with 5 minutes interval between each administration.

Group 5 (negative control group): Animals were treated with 1 drop of PBS then, 5 minutes later, 1 drop of 0.1% dexamethasone (Maxidex®).

On Day 5, 30 minutes after the last assessment of tolerability, the test, control or reference items were instilled in both eyes (1 drop in each eye). Subsequently, rabbits were euthanized and both eyes were enucleated and corneas were harvested and stored at −80° C. until analysis.

TABLE 2
Treatment received per animal group.

| Group No | Rabbit No | Left Eye | Right Eye |
|---|---|---|---|
| 1 | 01-10 | Control | 0.1% SPL micelles + 0.1% DXM |
| 2 | 11-20 | Control | 0.01% SPL micelles + 0.1% DXM |
| 3 | 21-30 | Control | 0.1% CANK + 0.1% DXM |
| 4 | 31-40 | Control | PBS |
| 5 | 41-50 | Control | 0.1% DXM |

2.2.4.4. Corneal Re-Epithelialization Evaluation

The size of the corneal wound was evaluated using the fluorescein test immediately after ocular debridement and once a day before the first instillation of the day. A baseline was recorded before the de-epithelialization. After instillation of a drop of fluorescein in the right (lesioned) eyes, the cornea was illuminated with blue light. Images of the corneal lesion (area stained by fluorescein) were taken using a CCD camera and analyzed using image J software.

2.2.4.5. Ocular Tolerability Examination

An ophthalmoscope was used for accurate examination of the conjunctiva, cornea and iris. Both eyes of each rabbit were examined using the ophthalmoscope during the pre-test period (baseline), then once daily after the last administration of the day from Day 0 to Day 4. The observations was scored using the Draize scale (Table S1).

2.2.4.6. Animal Sacrifice and Sampling

At the end of the measurement period, animals were euthanized by an intracardiac injection of overdosed pentobarbital following anaesthesia obtained by an intramuscular injection of ketamine-xylazine mixture. This method is one of the recommended methods for euthanasia by the European authorities. Immediately after euthanasia, both eyes were enucleated and corneas were dissected and stored at −80° C. until analysis.

2.2.4.7. Extraction and Quantification of the Drugs in the Corneas

The extraction method of the drugs from the cornea was validated and will be published separately (manuscript submitted). The 50 treated and 50 control corneas stored at −80° C. were thawed at room temperature, weighed and ground manually into small pieces which were placed in a glass vial containing 1 mL of MeOH:H₂O (1:1) and 100 ng/mL internal standard (IS, 17α-methyltestosterone). The vials were left under stirring at 300 rpm overnight for extraction. The following day, samples were centrifuged during 20 min at 12 000 rpm and the supernatants were quantified using the validated UHPLC-MS method.

2.2.5. Statistical Analysis

Statistical analysis on the percentages of re-epithelialization of the groups was performed using Kruskal-Wallis one-way analysis of variance on ranks followed by Student-Newman-Keuls post-hoc analysis. Statistical analysis on the mean concentrations found in the left and right corneas was performed using Student t-test or Mann-Whitney rank sum test.

### 3. RESULTS

3.1. Spironolactone Micelles Formulation and Optimization

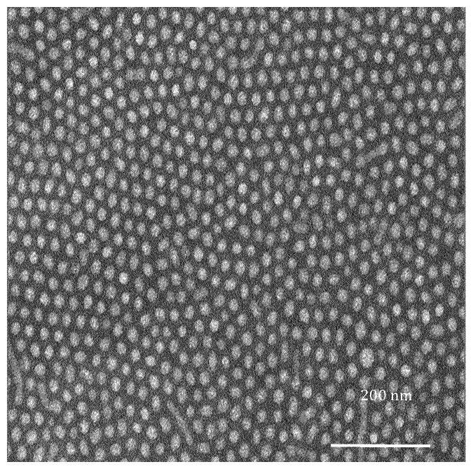

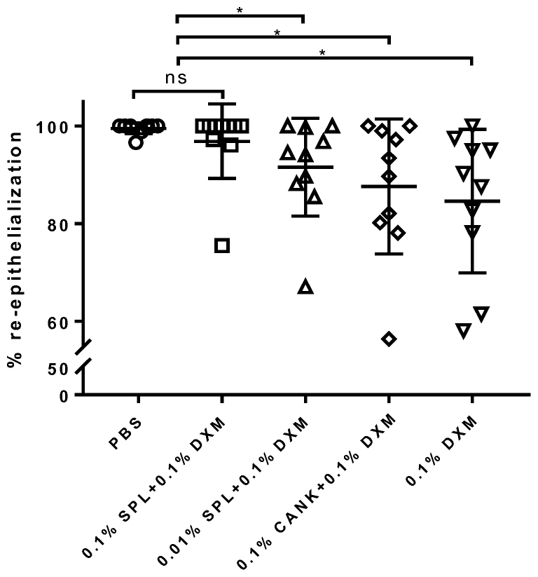

The incorporation efficiency of SPL in the mPEG-hexPLA micelles varied according to the SPL:copolymer ratio and the buffer used. Overall, the formulation containing SPL and mPEG-hexPLA copolymer at a ratio of 1:40 and citrate buffer (10 mM, pH 5.5) as the aqueous phase (Formulation A) achieved the best incorporation efficiency of 99.2±0.2% corresponding to a drug loading of 24.8±0.1 mg/g (Table 3). In the case of PBS (10 mM, pH 7.4), the best incorporation efficiency of 83.6±0.6% was achieved with SPL:copolymer ratio of 1:60 (Formulation B) corresponding to a drug loading of 14.0±0.1 mg/g (Table 3). The intensity weighted micelle diameters (Z-average, Zₐᵥ) were 50 and 52 nm and the number weighted micelle diameters (dₙ) were 17 and 19 nm (PDI=0.2) for Formulation A and B, respectively (Table 3). FIG. 1 shows the spherical and homogeneous aspect of the nanocarriers.

FIG. 1 shows a transmission electron microscopy (TEM) image of 0.1% spironolactone loaded micelles. Average particle size: 20 nm.

The stability of Formulations A and B was monitored during one month at 5° C. Results showed that Formulation A remained stable over one month in terms of its concentration, pH and osmolarity versus two weeks for Formulation B. Formulation A was optimized to achieve 100% incorporation efficiency corresponding to SPL concentration of 1 mg/mL. It was noticed that a small amount of SPL and mPEG-hexPLA was lost in the syringe during addition to the aqueous solution; to correct this, it was decided to prepare the formulation with 10% excess (by weight) of spironolactone and mPEG-hexPLA, corresponding to the amount lost during the formulation process. Given the incorporation efficiency and the superior stability, Formulation A was selected as the lead formulation to be used in further studies.

TABLE 3
Characterization of the different formulations.

| | | Target | [SPL]_{t0 ±} | Target | | | | | |
|---|---|---|---|---|---|---|---|---|---|
| | | [SPL]^{b} | SD^{c} | DL^{d} | DL ± SD | IE^{e} ± SD | Size (nm) | | |
| Buffer | Ratio^{a} | (mg/mL) | (mg/mL) | (mg/g) | (mg/g) | (%) | dₙ | Zₐᵥ | PDI |
| Citrate | 1:20 | 1 | 0.49 ± 0.01 | 50.0 | 24.5 ± 0.5 | 49.0 ± 1.0 | 13 | 53 | 0.2 |
| | 1:40 | 1 | 0.89 ± 0.00 | 25.0 | 22.3 ± 0.1 | 89.1 ± 0.2 | 17 | 50 | 0.2 |
| | 1:60 | 1 | 0.85 ± 0.01 | 16.7 | 14.2 ± 0.1 | 84.8 ± 0.7 | 16 | 50 | 0.2 |
| PBS | 1:20 | 1 | 0.45 ± 0.02 | 50.0 | 22.4 ± 1.0 | 44.8 ± 2.0 | 31 | 55 | 0.2 |
| | 1:40 | 1 | 0.73 ± 0.00 | 25.0 | 18.2 ± 0.1 | 72.9 ± 0.3 | 25 | 56 | 0.2 |
| | 1:60 | 1 | 0.84 ± 0.01 | 16.7 | 14.0 ± 0.1 | 83.6 ± 0.6 | 19 | 52 | 0.2 |

^{a}SPL: copolymer ratio,
^{b}concentration of SPL,
^{c}standard deviation,
^{d}drug loading,
^{e}incorporation efficiency.

3.2. Characterization of the Formulations Used in the In Vivo Study

Formulation characteristics are summarized in Table 4. The stability of the 0.01% and 0.1% spironolactone micellar formulations was assessed over 6 months at 5° C. Concentration, pH and particle size remained perfectly stable over the 6 month period. The stability of the 0.1% potassium canrenoate solution was assessed over 24 days at 5° C., to ensure product stability for the duration of the animal study. Again, the concentration and pH remained stable over the studied period.

TABLE 4
Characterization of the formulations used during the in vivo study.

| | Conc.^{a} ± SD^{b} | | Size (nm) | | |
|---|---|---|---|---|---|
| Formulation | (mg/mL) | pH | dₙ | Zₐᵥ | PDI |
| 0.1% SPL micelles | 1.03 ± 0.00 | 5.5 | 20 | 48 | 0.2 |
| 0.01% SPL micelles | 0.10 ± 0.00 | 5.5 | 26 | 49 | 0.2 |
| 0.1% CANK solution | 0.95 ± 0.00 | 8.0 | — | — | — |

^{a}Measured concentration of spironolactone (SPL) or potassium canrenoate (CANK),
^{b}standard deviation.

3.3. Tolerability and Efficacy Study in New Zealand White Rabbits

3.3.1. Ocular Tolerability

Ocular examinations of the animals on Day 4 are reported per treatment group in Table 5. Conjunctival redness, chemosis, discharge, iritis and corneal opacities were scored according to the Draize scale (Table S2). Most of the ocular reactions observed were slight and transient and were not attributed to the treatment since they are commonly observed in the de-epithelialization model. No ocular reaction was observed on Day 5 for all the groups except for one animal treated with DXM alone which still displayed a slight conjunctival redness (score 1 on a scale up to 3), a mild chemosis (score 2 on a scale up to 4) associated to a moderate discharge (score 2 on a scale up to 3) on Day 5. Indeed, this animal still exhibited a marked corneal re-epithelialization defect on Day 5 (−42.6%), this having possibly contributed to a persistent ocular reaction.

TABLE 5
Ocular observations of the animals on Day 4. Score
(italic) and number of animals concerned.

| | 0.1% SPL | 0.01% SPL | | | |
|---|---|---|---|---|---|
| | micelles + | micelles + | 0.1% CANK + | | 0.1% |
| | 0.1% DXM | 0.1% DXM | 0.1% DXM | PBS | DXM |
| Conjunctival | 1/3** | 1/3** | 1/3** | * | 1/3** |
| redness | 1/10 | 2/10 | 5/10 | | 4/10 |
| Chemosis | 1/4 | * | * | * | 2/4 |
| | 1/10 | | | | 1/10 |
| Discharge | * | * | 1-2/3 | * | 2/3 |
| | | | 5/10 | | 1/10 |
| Iritis | * | * | 1/2** | * | * |
| | | | 2/10 | | |
| Corneal | *** | Intensity: 1/4 | Intensity: 1-2/4 | Intensity: 1/4 | Intensity: 1/4 |
| opacities | | Area: 1-2/4 | Area: 1-4/4 | Area: 1/4 | Area: 1/4 |
| | | 4/10 | 10/10 | 5/10 | 4/10 |

* No reaction was observed,
**Observation concerns the right treated eye only,
*** Corneal opacities were observed but not scored. Conjunctival redness: conjunctival hyperemia, chemosis: swelling of the bulbar conjunctiva, discharge: mucus, pus or excessive tearing from the eye, iritis: inflammation of the iris, corneal opacities: loss of the cornea transparency. Scoring according to the Draize scale.

3.3.2. Corneal Wound Healing

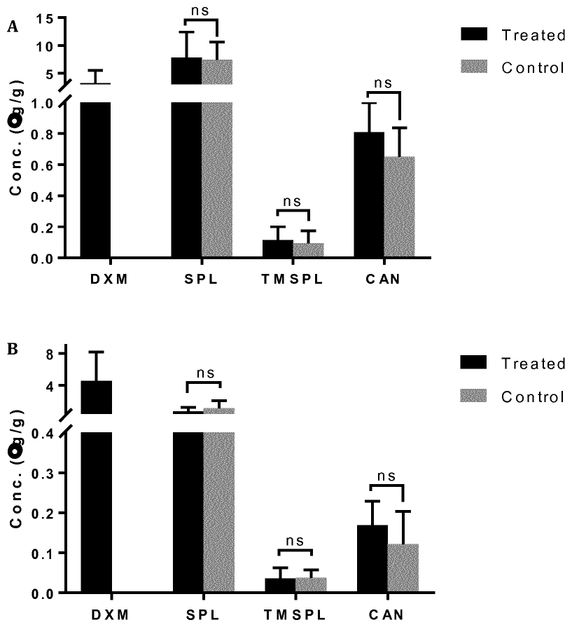

A significant beneficial effect of the 0.1% spironolactone micelles on the corneal epithelial wound healing was observed from Day 4. The mean percentages of re-epithelialization achieved on Day 4 according to the treatment received are shown in FIG. 2.

As expected, re-epithelialization of the wounded corneas treated with 0.1% DXM (Maxidex®) was delayed compared to the corneas treated with PBS alone. In this model, we expected a 2-fold delay in the healing of the wounded area between 0.1% DXM and PBS on Day 2 or 3. This difference was observed on Day 3 with a percentage wounded area of 21.2±8.7% for the animals treated with 0.1% DXM versus 9.3±7.9% for the animals treated with PBS, which validated the model used in this study (Table S2).

After multiple topical administrations of 0.1% SPL micelles together with 0.1% DXM, a significant suppression of the dexamethasone-induced corneal delayed wound healing was observed on Day 4 with a mean percentage of re-epithelialization of 96.9±7.3% versus 84.6±13.9% with 0.1% DXM alone (p<0.05). Moreover, the percentage re-epithelialization achieved with co-administration of 0.1% SPL micelles with 0.1% DXM was statistically equivalent (p>0.05) to the positive control (PBS treatment alone—99.5±1.0%). Thus, 0.1% SPL micelles seemed to completely compensate the negative impact of 0.1% DXM on corneal re-epithelialization. This was confirmed when considering individual results within groups that clearly showed a high proportion of individuals with marked corneal re-epithelialization defects in the 0.1% DXM group in contrast to the animals in the groups receiving PBS alone or co-administration of 0.1% SPL micelles (FIG. 2).

After multiple topical administrations of 0.01% SPL micelles or 0.1% CANK solution together with 0.1% DXM, a trend towards a reduction in the impact of DXM on re-epithelialization was observed. Although the mean extents of re-epithelialization of the wounded area observed upon co-treatment with either 0.01% SPL micelles or 0.1% CANK solution remained higher than 0.1% DXM alone at Day 4 (91.6±9.5% and 87.6±13.1%, respectively versus 84.6±13.9%), these differences were not statistically significant (p>0.05). Therefore, in this model and with these study conditions, effects on re-epithelialization of 0.01% SPL micelles and 0.1% CANK solution treatments did not appear as evident as was the case for 0.1% SPL micelles (FIG. 2).

FIG. 2: Mean percentage re-epithelialization of the corneal wounds per treatment group after 4-days' treatment. Bars represent means, errors bars represent standard deviation. p-values were calculated using Kruskal-Wallis one-way analysis of variance on ranks followed by Student-Newman-Keuls post-hoc analysis test; ns (p>0.05), non-significant difference, * (p<0.05), significant difference.

3.3.3. Biodistribution and Quantification of the Drugs in the Cornea

Group 1: 0.1% Spironolactone Micelles+0.1% Dexamethasone (Maxidex®)

Multiple ocular instillation of 0.1% SPL micelles and 0.1% DXM to the right eyes of 10 animals during 5 days resulted in the detection in the right corneas of spironolactone and its metabolites, 7α-thiomethylspironolactone and canrenone, with mean concentrations of 7802±4387 ng/g, 114±82 ng/g and 809±180 ng/g, respectively. Dexamethasone was also detected in the right corneas with a concentration of 3233±2190 ng/g

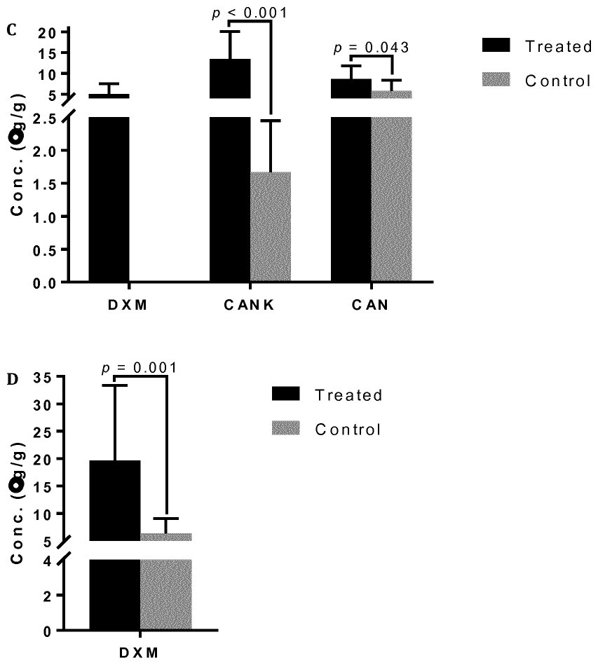

In Figure A). Interestingly, SPL and its metabolites were also detected in the left (control) corneas of all the animals instilled with 0.1% SPL micelles and 0.1% DXM with mean concentrations of 7406±3040 ng/g, 95±75 ng/g and 651±177 ng/g respectively for SPL, TMSPL and CAN. No significant difference in their mean concentrations was found between the treated and the control corneas (p>0.05). However, unlike the aforementioned molecules, DXM was not detected in the left corneas (FIG. 3A).

Group 2: 0.01% Spironolactone Micelles+0.1% Dexamethasone (Maxidex®)

Ocular instillation of 0.01% SPL micelles and 0.1% DXM to the right eyes of 9 animals (according to the Grubbs test, rabbit number 18 was an outlier and was excluded from the data analysis for Group 2) during 5 days resulted in the detection of SPL in the right corneas with a mean concentration of 715±488 ng/g, i.e. 10-fold less than the mean concentration found with 0.1% SPL micelles (7802±4387 ng/g) (FIG. 3B). The metabolites, TMSPL and CAN, were also detected at 36±25 ng/g and 168±57 ng/g, respectively, as was DXM (4542±3428 ng/g). As for the 0.1% SPL formulation, SPL and its metabolites were also detected in the left corneas of all the animals instilled with 0.01% SPL micelles and 0.1% DXM at concentrations of 1148±864 ng/g, 37±19 ng/g and 122±77 ng/g, respectively for SPL, TMSPL and CAN, with no significant difference in their mean concentrations between the treated and the control corneas (p>0.05). As for the animals in Group 1, DXM was again not detected in the left corneas (FIG. 3B).

Group 3: 0.1% Potassium Canrenoate Solution+0.1% Dexamethasone (Maxidex®)

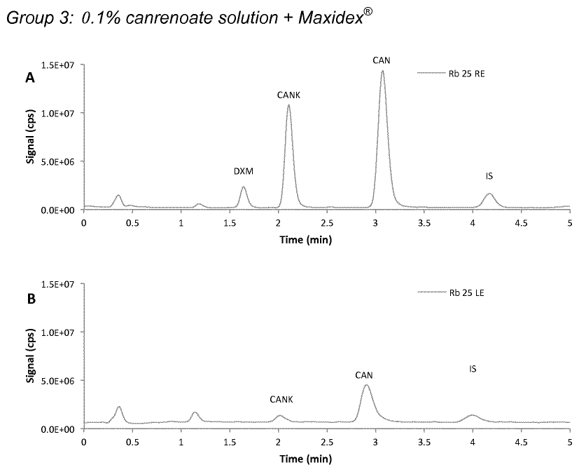

Ocular multiple instillation of 0.1% CANK solution and 0.1% DXM to the right eyes of 10 animals during 5 days allowed the detection of potassium canrenoate and canrenone at 13440±6346 ng/g and 8596±3097 ng/g, respectively, whereas dexamethasone was detected at a concentration of 5004±2376 ng/g (Figure C). CANK and CAN were also detected in the left corneas at 1672±739 ng/g and 6349±2379 ng/g, respectively, with a significant difference compared to the right corneas (p<0.05). Unlike in the right corneas, mean concentration of CAN was higher than that of CANK. DXM was not detected in the left corneas (FIG. 6 (3C)).

Group 4: PBS (Positive Control)

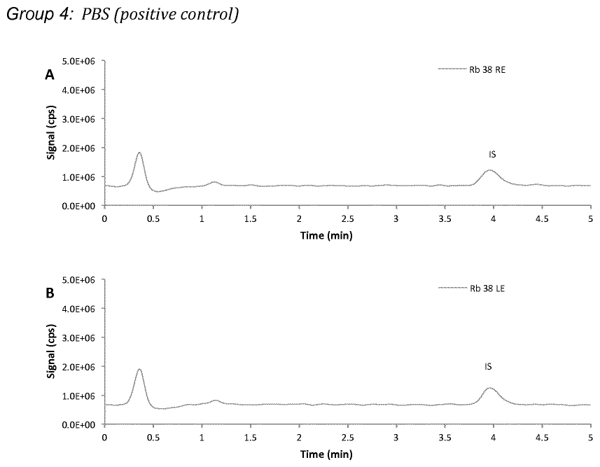

No drug was detected in the corneas obtained from the PBS treated animals (FIG. 7 (S4)).

Group 5: 0.1% Dexamethasone (Maxidex®—Negative Control)

Multiple ocular instillation of 0.1% DXM to the right eyes of 10 animals during 5 days resulted in the detection of dexamethasone in the right corneas at 19651±13032 ng/g. Interestingly, unlike for Groups 1-3, DXM was also detected in the left corneas at 6337±2603 ng/g (FIG. 3D) with a statistically significant difference compared to the right corneas (p<0.05).

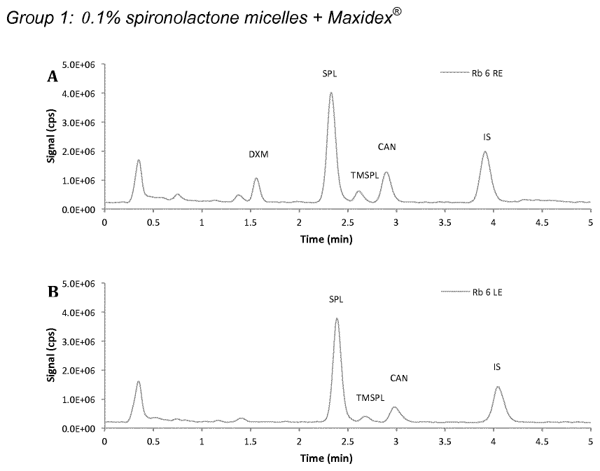

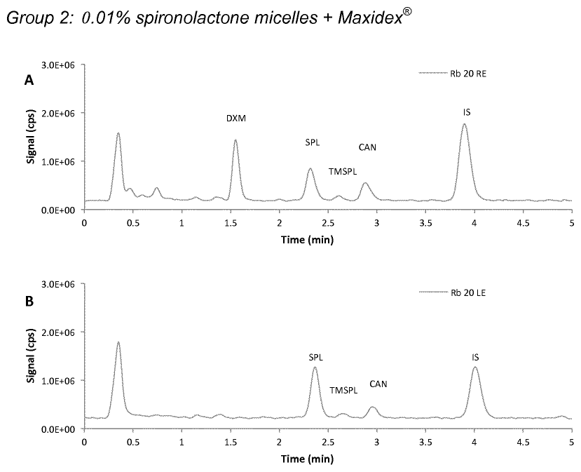

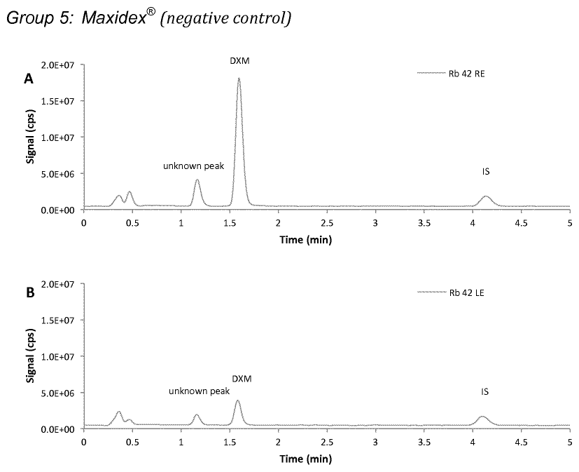

Typical chromatograms obtained from the analysis of both corneas from each group are provided in the supplementary data (FIGS. 4 to 8 (S1-5)).

In FIG. 3A-D: Mean concentrations of the drugs found in the right (treated) and left (control) corneas after 5-days multiple instillation. A: 0.1% spironolactone micelles followed by 0.1% dexamethasone (n=10); B: 0.01% spironolactone micelles followed by 0.1% dexamethasone (n=9); C: 0.1% potassium canrenoate solution followed by 0.1% dexamethasone (n=10); D: 0.1% dexamethasone (n=10). p-values are obtained with Student t-test; ns: p>0.99.

### 4. SUMMARY OF EXAMPLES

The results of the in vivo study showed a significant beneficial effect of the 0.1% spironolactone micellar formulation on dexamethasone-induced delayed corneal wound healing and a good tolerability. Comparison of the mean SPL concentrations found in the corneas treated with the 0.1% and 0.01% SPL micellar formulations showed a 10-fold difference (7802±4387 ng/g and 715±488 ng/g, respectively), which is consistent with the 10-fold difference in the applied dose. These results show that there is a correlation between the applied SPL dose and the SPL amount quantified in the corneas pointing to the controlled delivery of SPL by the micelles. In addition to the quantification of the drugs in the corneas, the biodistribution study provided information on their metabolism in the eye, and to a certain extent, on their mechanism of action. Indeed, multiple topical instillation of spironolactone to the eye resulted in the detection of its two main metabolites i.e. 7α-thiomethylspironolactone and canrenone, confirming the presence of thioesterase and thiol methyltransferase activity in the rabbit eye. The detection of canrenone after multiple topical instillation of potassium canrenoate confirmed the in situ conversion of canrenoate to canrenone via lactonization of the g-hydroxy acid group and so confirming the presence of paraoxonase enzyme (PON) in the rabbit eye.

Table 6 summarizes the mean concentrations of SPL, TMSPL and CAN found in the right (treated) corneas following multiple topical instillation of 0.1% SPL micelles, 0.01% SPL micelles or 0.1% CANK solution and their corresponding mean percentage of re-epithelialization. The difference in the mean percentage of re-epithelialization obtained with 0.1% SPL micelles was superior and significantly different from the mean percentage re-epithelialization obtained with 0.01% SPL micelles and 0.1% CANK solution (p<0.05); however, there was no significant difference in the mean percentage of re-epithelialization obtained between the latter two groups (p>0.05). The highest CAN concentration level was found in the corneas treated with 0.1% CANK; however, these corneas had the lowest percentage of re-epithelialization, suggesting that CAN is not the main metabolite involved in the mineralocorticoid receptor antagonism upon application of SPL. The mean percentages of re-epithelialization achieved with 0.1% and 0.01% SPL micelles were higher, supporting the higher potency of TMSPL over CAN as a mineralocorticoid receptor antagonist and evidencing its ability to counter-act the GC side effects and thus improve wound healing. These findings are consistent with previously published data: (i) Corvol et al. [19] pointed out the importance of the C₇ side chain for MR antagonism and reported a 10-fold lower CAN affinity for the MR as compared to SPL (and its sulfur-containing metabolite i.e. TMSPL), and a very low affinity of CANK for the MR since the negative charge of the carboxylate hinders binding to the receptor as there is no compensatory positive charge in the vicinity; (ii) Sutanto et al. [20] reported the higher potency of SPL compared to CANK with half maximal inhibitory concentrations (IC₅₀) of 4.9 nM and >1000 nM, respectively.

TABLE 6
Mean concentrations of SPL, TMSPL and CAN found in the treated corneas
at Day 5 and their corresponding percentage of re-epithelialization
at Day 4, following multiple instillation of 0.1% SPL micelles (n
= 10), 0.01% SPL micelles (n = 9) and 0.1% CANK solution (n = 10).

| | Mean concentration in the treated corneas ± SD (ng/g) | | | % re-epithe- |
|---|---|---|---|---|
| | SPL | TMSPL | CAN | lialization |
| 0.1% SPL micelles | 7802 ± 4387 | 114 ± 82 | 809 ± 180 | 96.9 ± 7.3 |
| 0.01% SPL micelles | 715 ± 488 | 36 ± 25 | 168 ± 57 | 91.6 ± 9.5 |
| 0.1% CANK | — | — | 8596 ± 3097 | 87.6 ± 13.1 |

Detection of the drugs in the contralateral eye During this study, animals received the different treatments only in the right eye, the left eye was kept as a control. Interestingly, after multiple instillation of the different treatments, SPL, CANK and their metabolites were detected in the left (control) corneas of all the treated animals. More interesting, DXM was only detected in the left (control) corneas of the animals that did not receive any MR antagonists (Group 5).

It has been reported that unilateral ocular administration of a drug leads to its detection in the contralateral eye [21-24] and this was explained in two different ways. The first involves a local non-hematogenous route where a direct passage from one eye to another can occur, especially in rats and lagomorphs, by interorbital communication either via lymphatic spread or via the lacrimal duct system with retrograde flow into the uninstilled eye [22]. Indeed, a previous study confirmed clinically and histologically the conjunctival cross-transfer of an antigen in rabbits using labelled human serum albumin [23]. In another study, iontophoresis of glucocorticoids into rat eyes, resulted in the observation of GC effects in the contralateral eye at levels much higher than those deemed compatible with systemic passage [24]. The second explanation involves the hematogenous route, which involves the return of the drug to the eyes through the general circulation. Indeed, after topical instillation of a drug, there are two main pathways of entry into the anterior segment: (i) across the cornea and (ii) across the conjunctiva. When the drug is crossing the conjunctiva, a fraction of the drug will be lost into the conjunctival blood circulation and the rest will diffuse into the sclera before reaching the heavily vascularized choroid, where another part is also cleared into the general circulation. This phenomenon is particularly significant in rabbits but is unlikely to be of importance in humans [21]. The possibility of external contact transfer of the molecules from one eye to another with the rabbit paw was excluded regarding the equivalent concentrations of SPL and its metabolites found in both eyes in all the animals.

Another interesting observation was the comparison between the concentrations found in the right (treated) eye versus the left (control) eye according to each treatment and each drug. Indeed, in Group 1 and 2, concentrations of SPL, TMSPL and CAN in the treated and control corneas were statistically equivalent; however, DXM was only detected in the treated corneas. In Group 3, concentrations of CANK and CAN in the treated and control corneas were statistically different (13.44±6.35 μg/g vs 1.67±0.74 μg/g and 8.60±3.10 μg/g vs 5.84±2.38 μg/g for CANK and CAN, respectively). It should be noted that the mean concentration of CANK is higher than CAN in the treated eye, whereas the opposite is the case for the contralateral eye. CANK once administered is available in the body as canrenoic acid, which is in equilibrium with its metabolite, canrenone. Indeed, the g-hydroxy acid on the C₁₇ of CANK is converted by cyclization to the g-lactone present in CAN by the paraoxonase enzyme (PON). Our findings confirm the presence of PON in the rabbit eye; however, the higher mean concentration of CAN compared to CANK found in the contralateral eye suggests that PON in the plasma and/or other tissues play a significant role in the biotransformation of CANK to CAN, resulting in the higher levels of CAN found in the contralateral eye. As in Group 1 and 2, DXM was only detected in the right treated corneas.

Mineralocorticoid receptor antagonists prevented dexamethasone binding to the MR In Group 5, unlike in Group 1-3, DXM was detected in both the treated and control corneas, although levels in the control corneas were significantly lower (p=0.001). Interestingly, DXM mean concentration in the treated corneas was found to be at least 4-fold higher in the absence of any MR antagonist (19.65±13.03 μg/g vs 3.23±2.19 μg/g, 4.54±3.43 μg/g and 5.00±2.38 μg/g for Group 5, 1, 2 and 3, respectively). These findings demonstrate that DXM binding to the MR was prevented by the presence of a MR antagonist (SPL, TMSPL, CAN or CANK).

This can be explained by the saturation of MR by the MR antagonist (SPL, TMSPL, CAN or CANK) in Group 1-3, leading to a lower occupancy of the MR by DXM, which is consequently eliminated more quickly given the relative short plasma half-life of DXM in rabbit estimated at 1.9 h [25] (cf. 1.4, 13.8 and 16.5 h respectively for SPL, TMSPL and CAN [26, 27]).

Moreover, Rafestin-Oblin et al. [28, 29] reported a higher affinity of SPL to MR (k_{d -}3.6 nM) compared to DXM (k_{d=}10 nM). Stokes et al. [30] reported that the MR concentration in the human corneal epithelium and endothelium is 3-times higher than the GR concentration. Given the above, the 4-fold higher DXM concentrations found in the treated corneas and its detection only in the contralateral corneas of the animals in Group 5 might be explained by the fact that in this case, there was no competition to bind to MR since there were no MR antagonists. Thus, the DXM mean concentration found in Group 5 was the sum of DXM bound to GR and to MR, whereas the mean concentrations found in Group 1, 2 and 3 corresponded to the unique fraction of DXM bound to GR. These findings confirm: (i) the increased off-target occupancy of MR by DXM in the absence of a MR antagonist and (ii) the resulting delayed wound healing when considering the percentage of re-epithelialization obtained with Group 5. Finally, the results support the rationale of using MR antagonist co-administration in conjunction with a prolonged GC therapy to prevent the delayed wound healing side-effect associated to the GC.

### 5. FINAL CONCLUSION

A stable spironolactone micellar formulation (0.1%, w/v) for topical administration was developed and tested in vivo in New Zealand white rabbits with respect to tolerability and efficacy in a corneal wound healing model.

The formulation was safe and showed beneficial effects on corneal wound healing management, i.e. the use of spironolactone micelles countered the delayed wound healing caused by glucocorticoid therapy. This is the first study showing that MR antagonism can efficiently prevent the epithelial healing delay induced by glucocorticoids, providing evidence that MR activation by glucocorticoids prevents epithelial growth and/or differentiation. MR antagonism may exert beneficial effects through modulation of several mechanisms known to be induced by MR activation, such as monocyte/macrophage and polymorphonuclear leukocyte activation, expression and activity of metalloproteinases, and expression of pro-fibrotic molecules [11, 17, 31]. MR could also directly influence the expression of ion channels such as ENAC and therefore influence epithelial cell migration [32]. It can be anticipated that in human respective results can be achieved with the new formulation according to the disclosure. Importantly, these preclinical in vivo results highlight the effect of the co-administration of the MR antagonist, spironolactone, in off-setting the glucocorticoid-induced delay in wound healing. Successful translation of these results to the clinic could improve therapeutic outcomes for glucocorticoid-treated patients since topical instillation of the spironolactone micelles might counter the impaired wound healing associated with routine glucocorticoid therapy.

Additional data show the advantages of the new formulation according to the disclosure:

Percentages of the Wounded Area Over Time

TABLE 7
Mean percentage wounded area over 5-days per treatment group.

| | | Wounded area (%) | | | | | |
|---|---|---|---|---|---|---|---|
| | | Day 0 just | | | | | |
| | | after the | | | | | |
| Treatment | | induction | Day 1 | Day 2 | Day 3 | Day 4 | Day 5 |
| 0.1% SPL micelles + | Mean | 100 | 81.0 | 37.9 | 11.9 | 3.1 | 1.8 |
| Maxidex ® | SD | 100 | 8.2 | 6.6 | 12.2 | 7.6 | 4.1 |
| 0.01% SPL micelles + | Mean | 100 | 84.6 | 35.3 | 13.1 | 8.4 | 3.7 |
| Maxidex ® | SD | 100 | 6.6 | 9.0 | 9.2 | 10.0 | 5.0 |
| 0.1% CANK solution + | Mean | 100 | 79.4 | 37.9 | 13.9 | 12.4 | 8.0 |
| Maxidex ® | SD | 100 | 7.6 | 10.4 | 10.0 | 13.8 | 9.1 |
| PBS | Mean | 100 | 80.7 | 38.0 | 9.3 | 0.5 | 0.0 |
| | SD | 100 | 6.9 | 10.2 | 8.3 | 1.1 | 0.0 |
| Maxidex ® (0.1% | Mean | 100 | 86.4 | 45.9 | 21.2 | 15.4 | 11.7 |
| dexamethasone) | SD | 100 | 6.9 | 10.0 | 9.2 | 14.7 | 15.1 |

Draize Scale

TABLE 8
Draize scale for ocular observations scoring.
CONJUNCTIVA

| | | |
|---|---|---|
| a. Chemosis | No swelling | 0 |
| (lids and/or | Slight swelling (incl. Nictitating membrane) | 1 |
| nictitating | Obvious visible swelling with eversion of lids | 2 |
| membrane) | Swelling which leads to half closed lids | 3 |
| | Swelling which leads to half closed lids, up to | 4 |
| | totally closed lids | |
| b. Discharge | No discharge | 0 |
| | Slight discharge (not including normal | 1 |
| | secretions) | |
| | Discharge with moistening of lids and hairs | 2 |
| | just adjacent to lids | |
| | Discharge with moistening of lids and hairs | 3 |
| | just adjacent to lids on a considerable area | |
| | around the eye | |
| c. Redness | Vessels normal | 0 |
| | Hyperaemia | 1 |
| | Diffuse redness, individual vessels not discernible | 2 |
| | Massive redness of all sections | 3 |

IRIS

| | | |
|---|---|---|
| d. Iritis | Normal | 0 |
| | Markedly deepened folds, congestion, swelling | 1 |
| | moderate circumcorneal injection (any of these | |
| | or combination of any thereof), iris still reacting | |
| | to light | |
| | No reaction to light, haemorrhage, gross | 2 |
| | destruction (any or all these) | |

CORNEA

| | | |
|---|---|---|
| e. Opacity | No opacity | 0 |
| | Scattered or diffuse areas of opacity, details | 1 |
| | of iris clearly visible | |
| | Not completely translucent areas, details | 2 |
| | of iris slightly obscured | |
| | Nacreous areas, details of iris not visible, size | 3 |
| | of pupil barely discernible | |
| | Complete corneal opacity, iris not discernible | 4 |
| f. Involvement | No involvement | 0 |
| of opacities | One quarter or less, but not 0 | 1 |
| | Exceeding one quarter, but less than half | 2 |
| | Exceeding one half, but less than three quarters | 3 |
| | Exceeding three quarters up to whole area | 4 |

### ABBREVIATIONS

CAN Canrenone
CANK Potassium canrenoate
dₙ Number weighted particle diameter
DXM Dexamethasone
GC Glucocorticoid
GR Glucocorticoid receptor
HSD2 11b-hydroxysteroid dehydrogenase type II
IC₅₀ Half maximal inhibitory concentration
K_{d} Dissociation constant at equilibrium
MC Mineralocorticoid
MeT 17α-methyltestosterone
mPEG-hexPLA Methoxy-poly(ethylene glycol)-hexyl-substituted-poly(lactic acid)
MR Mineralocorticoid receptor
MRA Mineralocorticoid receptor antagonist
PBS Phosphate buffered saline
PDI Polydispersity index
PON Paraoxonase
SD Standard deviation
SPL Spironolactone
TMSPL 7α-thiomethylspironolactone
UHPLC-ESI-MS Ultra-High Performance Liquid Chromatography coupled to Electrospray Ionization Mass Spectroscopy
WH Wound healing
Zₐᵥ Z-average, Intensity weighted particle diameter

### REFERENCES

[1] S. Werner, R. Grose, Regulation of wound healing by growth factors and cytokines, Physiol. Rev. 83 (2003) 835-870.
[2] V. T. Nguyen, N. Farman, E. Maubec, D. Nassar, D. Desposito, L. Waeckel, S. Aractingi, F. Jaisser, Re-epithelialization of pathological cutaneous wounds is improved by local mineralocorticoid receptor antagonism, J. Invest. Dermatol. 136 (2016) 2080-2089.
[3] S. C. Park, J. H. Kim, Effect of steroids and nonsteroidal anti-inflammatory agents on stromal wound healing following excimer laser keratectomy in rabbits, Ophthalmic Surg. Lasers 27 (1996) S481-486.
[4] M. Srinivasan, P. Lalitha, R. Mahalakshmi, N. V. Prajna, J. Mascarenhas, J. D. Chidambaram, S. Lee, K. C. Hong, M. Zegans, D. V. Glidden, S. McLeod, J. P. Whitcher, T. M. Lietman, N. R. Acharya, Corticosteroids for bacterial corneal ulcers, Br. J. Ophthalmol. 93 (2009) 198-202.
[5] S. Adams, R. S. Valchanova, B. Munz, RIP2: a novel player in the regulation of keratinocyte proliferation and cutaneous wound repair?, Exp. Cell Res. 316 (2010) 728-736.
[6] M. Liu, K. Saeki, T. Matsunobu, T. Okuno, T. Koga, Y. Sugimoto, C. Yokoyama, S. Nakamizo, K. Kabashima, S. Narumiya, T. Shimizu, T. Yokomizo, 12-Hydroxyheptadecatrienoic acid promotes epidermal wound healing by accelerating keratinocyte migration via the BLT2 receptor, Journal Experimental Medicine 211 (2014) 1063-1078.
[7] M. Kadmiel, A. Janoshazi, X. Xu, J. A. Cidlowski, Glucocorticoid action in human corneal epithelial cells establishes roles for corticosteroids in wound healing and barrier function of the eye, Exp. Eye Res. 152 (2016) 10-33.
[8] N. Hashizume, S. Saika, Y. Okada, T. Miyamoto, K. Shimizu, Y. Ohnishi, Effects of antiinflammatory drugs on migration of the rabbit corneal epithelium, J. Cataract Refract. Surg. 27 (2001) 1499-1502.
[9] E. Maubec, C. Laouenan, L. Deschamps, V. T. Nguyen, I. Scheer-Senyarich, A. C. Wackenheim-Jacobs, M. Steff, S. Duhamel, S. Tubiana, N. Brahimi, S. Leclerc-Mercier, B. Crickx, C. Perret, S. Aractingi, B. Escoubet, X. Duval, P. Arnaud, F. Jaisser, F. Mentre, N. Farman, Topical mineralocorticoid receptor blockade limits glucocorticoid-induced epidermal atrophy in human skin, J. Invest. Dermatol. 135 (2015) 1781-1789.
[10] A. A. Sarchahi, A. Maimandi, A. K. Tafti, M. Amani, Effects of acetylcysteine and dexamethasone on experimental corneal wounds in rabbits, Ophthalmic Res. 40 (2008) 41-48.
[11] C. S. De Paiva, R. M. Corrales, A. L. Villarreal, W. J. Farley, D. Q. Li, M. E. Stern, S. C. Pflugfelder, Corticosteroid and doxycycline suppress MMP-9 and inflammatory cytokine expression, MAPK activation in the corneal epithelium in experimental dry eye, Exp. Eye Res. 83 (2006) 526-535.
[12] O. Stojadinovic, L. E. Lindley, I. Jozic, M. Tomic-Canic, Mineralocorticoid Receptor Antagonists-A New Sprinkle of Salt and Youth, J. Invest. Dermatol. 136 (2016) 1938-1941.
[13] G. Petroutsos, R. Guimaraes, J. P. Giraud, Y. Pouliquen, Corticosteroids and corneal epithelial wound healing, Br. J. Ophthalmol. 66 (1982) 705-708.
[14] P. Fagerholm, Wound healing after photorefractive keratectomy, J. Cataract Refract. Surg. 26 (2000) 432-447.
[15] A. S. Wang, E. J. Armstrong, A. W. Armstrong, Corticosteroids and wound healing: clinical considerations in the perioperative period, Am. J. Surg. 206 (2013) 410-417.
[16] J. K. Youm, K. Park, Y. Uchida, A. Chan, T. M. Mauro, W. M. Holleran, P. M. Elias, Local blockade of glucocorticoid activation reverses stress- and glucocorticoid-induced delays in cutaneous wound healing, Wound Repair Regen. 21 (2013) 715-722.
[17] N. Farman, V. T. Nguyen, A novel actor in skin biology: the mineralocorticoid receptor, Exp. Dermatol. 25 (2016) 24-25.
[18] J. Boix, L. M. Sevilla, Z. Saez, E. Carceller, P. Perez, Epidermal mineralocorticoid receptor plays beneficial and adverse effects in skin and mediates glucocorticoid responses, J. Invest. Dermatol. DOI 10.1016/j.jid.2016.07.018 (2016).
[19] P. Corvol, M. Claire, M. E. Oblin, K. Geering, B. Rossier, Mechanism of the antimineralocorticoid effects of spirolactones, Kidney Int. 20 (1981) 1-6.
[20] W. Sutanto, E. R. de Kloet, Mineralocorticoid receptor ligands: biochemical, pharmacological, and clinical aspects, Med. Res. Rev. 11 (1991) 617-639.
[21] D. M. Maurice, S. Mishima, Ocular Pharmacokinetics, in: M. L. Sears (Ed.) Pharmacology of the Eye, Springer Berlin Heidelberg, Berlin, Heidelberg, 1984, pp. 19-116.
[22] P. R. Jensen, S. B. Aronson, M. Pollycove, E. Yamamoto, Mechanisms of host response in the eye. 3. Interocular protein transfer, Arch. Ophthalmol. 77 (1967) 814-817.
[23] P. Jensen, M. Pollycove, S. B. Aronso, E. Yamamoto, Radioisotopic measurements of protein transfer between the eyes, Invest. Ophthalmol. Vis. Sci. 3 (1964) 676.
[24] F. Behar-Cohen, J. M. Parel, Y. Pouliquen, B. Thillaye-Goldenberg, O. Goureau, S. Heydolph, Y. Courtois, Y. De Kozak, Iontophoresis of dexamethasone in the treatment of endotoxin-induced-uveitis in rats, Exp. Eye Res. 65 (1997) 533-545.
[25] P. E. Stokes, P. M. Stoll, J. H. Schluger, B. Lasley, Hypercortisolemia decreases dexamethasone half-life in rabbit, J. Psychiatr. Res. 36 (2002) 423-428.
[26] B. A. Maron, J. A. Leopold, Mineralocorticoid receptor antagonists and endothelial function, Curr Opin Investig Drugs 9 (2008) 963-969.
[27] J. Beyer, A. Bierl, F. T. Peters, H. H. Maurer, Screening procedure for detection of diuretics and uricosurics and/or their metabolites in human urine using gas chromatography-mass spectrometry after extractive methylation, Ther. Drug Monit. 27 (2005) 509-520.
[28] M. E. Rafestin-Oblin, M. Lombes, B. Couette, E. E. Baulieu, Differences between aldosterone and its antagonists in binding kinetics and ligand-induced hsp90 release from mineralocorticosteroid receptor, J. Steroid Biochem. Mol. Biol. 41 (1992) 815-821.
[29] M. E. Rafestin-Oblin, M. Lombes, P. Lustenberger, P. Blanchardie, A. Michaud, G. Cornu, M. Claire, Affinity of corticosteroids for mineralocorticoid and glucocorticoid receptors of the rabbit kidney: effect of steroid substitution, J. Steroid Biochem. 25 (1986) 527-534.
[30] J. Stokes, J. Noble, L. Brett, C. Phillips, J. R. Seckl, C. O'Brien, R. Andrew, Distribution of glucocorticoid and mineralocorticoid receptors and 1 lbeta-hydroxysteroid dehydrogenases in human and rat ocular tissues, Invest. Ophthalmol. Vis. Sci. 41 (2000) 1629-1638.
[31] F. Jaisser, N. Farman, Emerging Roles of the Mineralocorticoid Receptor in Pathology: Toward New Paradigms in Clinical Pharmacology, Pharmacol. Rev. 68 (2016) 49-75.
[32] H. Y. Yang, R. P. Charles, E. Hummler, D. L. Baines, R. R. Isseroff, The epithelial sodium channel mediates the directionality of galvanotaxis in human keratinocytes, J. Cell Sci. 126 (2013) 1942-1951.
[33]. Zhao M, Rodriguez-Villagra E, Kowalczuk L, Le Normand M, Berdugo M, Levy-Boukris R, El Zaoui I, Kaufmann B, Gurny R, Bravo-Osuna I, Molina-Martinez I T, Herrero-Vanrell R, Behar-Cohen F. J: Tolerance of high and low amounts of PLGA microspheres loaded with mineralocorticoid receptor antagonist in retinal target site Control Release. 2017 Nov. 28; 266:187-197

1. A pharmaceutical formulation comprising a spironolactone (also denoted: SC 9420; NSC-150339; 7α-Acetylthiospirolactone; 7α-Acetylthio-17α-hydroxy-3-oxopregn-4-ene-21-carboxylic acid γ-lactone) as well as tautomers, geometrical isomers, optically active forms, enantiomeric mixtures thereof, pharmaceutically acceptable salts and pharmaceutically active derivative thereof and at least one polymer or a polymer mixture of one or more of an alkyl substituted polylactide or/and a polymer prepared by melt polycondensation of one or more substituted or unsubstituted C₆-C₈ 2-hydroxyalkyl acid(s).

2. A pharmaceutical formulation according to claim 1 wherein the formulation can be sterile filtered.

3. A pharmaceutical formulation according to claim 1 wherein the formulation provides for improved tissue penetration characteristics of spironolactone.

4. A pharmaceutical formulation according to claim 1 wherein the spironolactone is spontaneously embedded into an amphiphilic shell.

5. A pharmaceutical formulation according to any of claims 1 to 4 wherein the polymer is selected from one or more of
a. one or more of a co-polymer consisting of mPEG and an alkyl substituted polylactide, and wherein the alkyl substituted polylactide is viscous and has the structure: 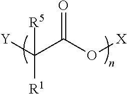
wherein R¹ is substituted or unsubstituted C₂-C₃₀ alkyl, wherein n is at least 2: and wherein R³ is hydrogen or substituted or unsubstituted alkyl. In specific aspects, the polymer can be a polymer of any one or more of the C₄-C₃₂ 2-hydroxylalkyl acids: wherein X is hydrogen or —C(O)—CH—CH2; and Y is selected from the group consisting of —OH, an alkoxy, benzyloxy and —O—(CH2-CH2-O)p-CH3; and wherein p is 1 to 700 and as disclosed in WO2007/012979 A1
and/or
b. one or more polymers prepared by melt polycondensation of one or more substituted or unsubstituted C₆-C₈ 2-hydroxyalkyl acid(s) as disclosed in WO2012/014011 A1.

6. A pharmaceutical formulation according to claims 1 to 5 wherein the active compound is a spironolactone and the polymer is a co-polymer consisting of mPEG and poly(caprylic acid).

7. A pharmaceutical formulation according to claims 1 to 6 for use in the preventing, repressing or treating a disease or disorder selected from the group comprising an ophthalmic disease or disorder, recurrent corneal erosions, wound healing delay particularly but not only due to association with the use of glucocorticoids, post surgical treatment of corneal graft or refractive surgery, or any other corneal surgery to favor re-epithelialization, glucocorticoid topical administration on desepithelialized cornea such as cornea traumatism, post corneal surgery, post cross-linking, post refractive surgery (laser assisted or surgical procedure), corneal dystrophies, ocular rosacea, corneal abscess or bacterial infection in association with antibiotics, corneal fibrosis and scaring because of the anti-fibrotic effects of spironolactone, corneal opacification, peripheral ulcerative keratitis, corneal neovascularization (due to anti-angiogenic effects of spironolactone), meibomian gland dysfunction and associated diseases such as dry eye syndroms and blepharitis.

8. A pharmaceutical formulation for use according to claim 7 wherein the use is characterized by a reduced incidence of side effects.

9. A method of preventing, repressing or treating a disease or disorder selected from the group comprising an ophthalmic disease or disorder, or recurrent corneal erosions, wound healing delay associated with the use of glucocorticoids, post surgical treatment of corneal graft to favor reepithelialization, glucocorticoid topical administration on desepithelialized cornea such as cornea traumatism, post corneal surgery, post cross-linking, post refractive surgery (laser assisted or surgical procedure), corneal dystrophies, ocular rosacea, corneal abscess association with antibiotics, corneal fibrosis and scaring due to anti-fibrotic effects of spironolactone, corneal opacification in a subject said method comprising administering to a subject in need thereof a pharmaceutical formulation according to any of claims 1 to 6.

10. A method for treating or preventing an ophthalmic disease or disorder associated with excessive stimulation of the mineralocorticoid receptor by administering a pharmaceutical formulation according to any of claims 1 to 6.

11. A method for treating an ophthalmic disease or disorder wherein the stimulation is engendered by corticosteroid therapy by administering a pharmaceutical formulation according to any of claims 1 to 6.

12. A method for treating an ophthalmic disease or disorder wherein the disease or disorder is selected from (list) by administering a pharmaceutical formulation according to any of claims 1 to 6.

13. A method for preparing a pharmaceutical composition according to any of claims 1 to 6 by mixing the two components at room temperature.

14. A formulation for use according to claim 7 or 8, or the method according to any of claim 9, 10, 11, or 12 for topical use or administration, or for a loco-regional use or administration.

15. A formulation for use according to any of claim 7 or 8, or a method according to any of claims 9 to 12 wherein it is used in patients with prior or concomitant treatment of gluco corticosteroids or gluco corticosteroid medication.
